(19) United States
(12) Patent Application Publication
(10) Pub. No.: US 2010/0331961 A1
(43) Pub. Date: Dec. 30, 2010

(54) STENT WITH IMPROVED STENT DESIGN

(76) Inventors: Nils Goetzen, Rostock (DE); Jan Schettler, Rostock (DE)

Correspondence Address: GREER, BURNS & CRAIN, 300 S WACKER DR, 25TH FLOOR, CHICAGO, IL, 60606, US

(21) Appl. No.: 12/821,753

(22) Filed: Jun. 23, 2010

Related U.S. Application Data
(60) Provisional application No. 61/220,212, filed on Jun. 25, 2009.

(51) Int. Cl. A61F 2/82

(52) U.S. Cl. 623/1.16

(57) ABSTRACT

An example embodiment of the invention is an expandable stent comprising a tubular base body with a lumen along a longitudinal axis, wherein the base body has a plurality of circumferential support structures which are successively positioned along the longitudinal axis and are each composed of a sequence of diagonal elements and arched elements, and has one or more connectors, wherein two successive circumferential support structures are joined together by at least one connector, and each connector is attached to one diagonal element of the two circumferential support structures to be connected; characterized in that a diagonal element to which a connector is attached has an elongated shape with two opposite ends, i) wherein the diagonal element at its first end has a branching point with a diameter d₁ at which the diagonal element directly branches into an arched element and a connector, ii) wherein the diagonal element at its second end has a connecting point with a diameter d₂ at which the diagonal element directly merges into a further arched element; and iii) wherein the ratio of d₁ to d₂>1, and the diagonal element is continuously tapered from its first end to its second end.

### CROSS REFERENCE

This application claims priority on U.S. Provisional Application No. 61/220,212 filed on Jun. 25, 2009.

### FIELD

One embodiment of the invention relates to a stent with an improved stent design.

### BACKGROUND

The implantation of stents has become established as one of the most effective therapeutic measures in the treatment of vascular diseases. One purpose of stents is to provide a support function in hollow organs of a patient. For this purpose stents of conventional design have a base body comprising a plurality of circumferential support structures composed of metallic struts, for example, which for introduction into the body are initially present in compressed form, then are expanded at the site of administration. One of the main fields of application of such stents is the permanent or temporary dilation and holding open of vascular constrictions, in particular constrictions (stenoses) of the coronary vessels. Also known are aneurysm stents, for example, which are used to support damaged vessel walls.

Stents have a tubular base body of sufficient load capacity to keep the constricted vessel open to the desired degree so that blood continues to flow through unhindered. The peripheral wall of the base body is generally formed from a lattice-like support structure which allows the stent, in a compressed state with a small outer diameter, to be inserted up to the constricted site to be treated in the vessel in question, and at that location to be expanded with the assistance of a balloon catheter, for example, until the vessel has the desired enlarged inner diameter. The process of positioning and expanding the stent during the procedure and the subsequent location of the stent in the tissue after the procedure is completed must be monitored by the cardiologist. This may be achieved using imaging methods such as X-ray analysis, for example.

The stent has a base body made of an implant material. An implant material is a nonliving material which is used for medical applications and interacts with biological systems. The basic requirement for use of a material as an implant material, which when properly used is in contact with the bodily surroundings, is compatibility with the body (biocompatibility). Biocompatibility is understood to mean the ability of a material to induce an appropriate tissue reaction in a specific application. This includes adaptation of the chemical, physical, biological, and morphological surface characteristics of an implant to the recipient tissue, with the objective of a clinically sought interaction. The biocompatibility of the implant material is also dependent on the time sequence of the reaction of the biosystem which has received the implant. Relatively short-term irritation and inflammation occur which may result in changes in the tissue. Accordingly, biological systems react in various ways, depending on the characteristics of the implant material. Implant materials may be divided into bioactive, bioinert, and biocorrodable/absorbable materials, depending on the reaction of the biosystem.

Some stents have a tubular base body which includes a lumen along a longitudinal axis. The base body has a plurality of circumferential support structures, for example circumferential cylindrical meandering rings or helices, successively positioned along the longitudinal axis. These support structures are each composed of a sequence of diagonal elements and arched elements (also referred to as crowns), and form the geometric base unit in modern stent designs. The support structures are joined together by connecting elements, so-called connectors, in the longitudinal direction. These connectors on the one hand must be situated so that they ensure sufficient bending flexibility of the stent, and on the other hand must not hinder a crimping and/or dilation process.

When magnesium, which does not have particularly favorable mechanical material properties, is used as a degradable stent material, minimal influences on the power flow in combination with effective utilization of the crimping space are of considerable importance, and thus require an optimal design of the connector attachment.

### SUMMARY

One feature of some embodiments of the present invention is to solve one or more of the problems described above. One example aim is to provide a stent design which allows only minimal influence of the power flow in the arched elements, with effective utilization of the space that is available for crimping.

The present disclosure provides, amongst other embodiments, an example embodiment of an expandable stent comprising a tubular base body with a lumen along a longitudinal axis, wherein the base body:
has a plurality of circumferential support structures which are successively positioned along the longitudinal axis and are each composed of a sequence of diagonal elements and arched elements; and
has one or more connectors, wherein two successive circumferential support structures are joined together by at least one connector, and each connector is attached to one diagonal element of the two circumferential support structures to be connected;
   characterized in that
   at least one diagonal element to which a connector is attached has an elongated shape with first and second opposite ends,
i) wherein the diagonal element at its first end has a branching point with a diameter dᵢ at which the diagonal element branches into an arched element and a connector; and
ii) wherein the diagonal element at its second end has a connecting point with a diameter d₂ at which the diagonal element merges into a further arched element; and
iii) wherein the ratio of dᵢ to d₂>1, and the diagonal element is tapered from its first end to its second end.

An additional embodiment of an expandable stent has a tubular base body made at least partially from a metallic implant material and has a lumen along a longitudinal axis. The base body comprises a plurality of circumferential support structures successively positioned along the longitudinal axis and each having a plurality of diagonal elements and a plurality of arched elements. At least one connector joins two successive circumferential support structures together through attachment to a diagonal element of each of the two circumferential support structures, the connector oriented in the longitudinal direction between the two connected circumferential support structures wherein in a dilated state of the stent the connector defines an angle α of between about 0° and about 70° with respect to the longitudinal axis. Each of the diagonal elements to which a connector is attached has an elongated and tapered shape between first and second opposing ends, the first end having a branching point with a diameter d₁ at which a diagonal element directly branches into an arched element and the connector, and the second end having a connecting point with a diameter d₂ at which a diagonal element directly merges into one of the arched elements, the ratio of d₁ to d₂ between about 1.1 and about 5.

### DESCRIPTION OF THE DRAWINGS

FIG. 1 schematically shows a section of a base body of a stent according to one invention embodiment; and

FIG. 2 schematically shows an enlarged section from FIG. 1, indicating the detailed shape of a diagonal element to which a connector is attached in one example embodiment.

### DETAILED DESCRIPTION OF EXAMPLE EMBODIMENTS

This application claims priority on U.S. Provisional Application No. 61/220,212 filed on Jun. 25, 2009; which application is incorporated herein by reference.

The approach according to some embodiments of the invention is characterized in that the transition region from the connector to the support structure extends over the entire region of a diagonal element, but does not extend into an arched structure. Due to the fact that the attachment of the connector is limited to one diagonal element and does not extend into an arched element, a homogeneous distribution of stresses and expansions in the arched elements of the stent remains unaffected. Homogeneous plastic deformability of the stent according to this embodiment of the invention is thus ensured. Because the connecting point between the connector and the diagonal element is located closely proximate to, or at an end of the diagonal element which is closer to the next support structure, the attachment occupies little space, thereby providing adequate room for ensuring sufficient crimpability and bending flexibility of the stent. As a result of the connection between the connector and the diagonal element being supported over substatially the entire length of, or over the entire length of the diagonal element, it has been discovered that the stent according to this invention embodiment has optimal transmission of force from one support structure to the next, as well as beneficial levels of stability. An orientation of the connectors essentially along the longitudinal axis and/or an acute-angled transition from the connector to the diagonal element result in optimal utilization of space in the crimped state of the stent according to some invention embodiments.

The expandable stent according to some invention embodiments has a tubular base body which encloses a lumen along a longitudinal axis. Blood is able to flow through this lumen after the stent has been provided in a blood vessel. The base body comprises a plurality of circumferential support structures, successively positioned along the longitudinal axis, which enclose the lumen. Each of the support structures is composed of a sequence of diagonal elements and arched elements. The diagonal elements have an elongated shape with two ends, and connect two arched elements having oppositely directed curvatures. The diagonal elements are essentially responsible for the extension of a support structure in the direction of the longitudinal axis. The arched elements are curved, and connect two successive diagonal elements of a support structure in such a way that the diagonal elements come to rest, one on top of the other, along an axis which extends vertically with respect to the longitudinal axis, resulting in an annular circumferential structure which encloses a lumen.

In addition to a plurality of support structures the base body includes one or more connectors, wherein two successive circumferential support structures are joined together by at least one connector. The connectors for the stent according to some embodiments are designed in such a way that a plurality of support structures may be connected to produce a base body which is suitable for use in an expandable stent. For this purpose, each connector is connected at a first end to a diagonal element of a first support structure, and at a second end is connected to a diagonal element of a second support structure. Two successive support structures may also be joined together by more than one connector. One, several, or all connectors of the stent may have an elongated shape with two opposite ends. The connectors are preferably only long enough to ensure sufficient flexibility of the two adjacent support structures, but not so long that the stent becomes torsionally flexible. One, several, or all connectors of a stent according to some invention embodiments may have a curved shape. One, several, or all connectors of a stent according to some invention embodiments may respectively branch off from the diagonal element at an acute angle. The connectors are basically oriented in the longitudinal direction between the two circumferential support structures to be connected, wherein the connectors do not necessarily have to be (but in some embodiments may be) in parallel alignment with the longitudinal axis. In the dilated state of the stent the connectors may form an angle α of ≧0° and <70°, preferably ≧0° and <45°, particularly preferably ≧0° and <25°, with respect to the longitudinal axis. Other values are contemplated and will prove useful in some other embodiments and applications. The “dilated state” may be understood to mean the state of the stent before the stent is crimped to a suitable application shape, as well as the state of the stent after the stent is implanted. The stent according to this embodiment has an angle α in the above-referenced ranges at least in one of the two forms of the dilated state.

The stent according to some invention embodiments has diagonal elements to which a connector is attached. A diagonal element to which a connector is attached has an elongated shape with two opposite ends. At its first end the diagonal element has a branching point with a diameter d₁. At the branching point the diagonal element merges directly into an arched element and a connector. At its second end the diagonal element has a connecting point with a diameter d₂. At the connecting point the diagonal element merges directly into a further arched element. The ratio of d₁ to d₂ in many embodiments is >1, and preferably is between 1.1 and 5, particularly preferably between 1.2 and 3, very particularly preferably between 1.25 and 2.5. Other values are contemplated and will prove useful in some other embodiments and applications, with two examples being the ratio set at greater than 5 and the ratio set below 1. The diagonal element tapers from its first end toward its second end. This tapering may be uniform. However, the tapering may also be nonuniform. In one example of a non-uniform tapering, the diagonal element is tapered more pronounced in a region of the diagonal element facing the first end than in a region facing the second end. In particular the attachment of the connector to the diagonal element in some embodiments is supported over the entire length of the diagonal element, and may have an organic design. For a stent according to this invention embodiment, one, several, or all connector connections between the support structures may be respectively made via the above-described diagonal elements.

The base body of the stent according to one invention embodiment may be composed of any implant material that is suitable for the manufacture of implants, in particular stents. Implant materials for stents include (but are not limited to) polymers, metallic materials, and ceramic materials. Biocompatible metals and metal alloys for permanent implants contain, for example, stainless steel (316L, for example), cobalt-based alloys (CoCrMo cast alloys, CoCrMo forged alloys, CoCrWNi forged alloys, and CoCrNiMo forged alloys, for example), pure titanium and titanium alloys (CP titanium, TiAl₆V₄, or TiAl₆Nb₇, for example), and gold alloys. The base body preferably contains a metallic implant material or is composed of same.

The stent according to some invention embodiments has a base body which contains a biodegradable implant material or is composed of same. For some biocorrodable stents of the invention the use of magnesium or pure iron, or biocorrodable base alloys of the elements magnesium, iron, zinc, molybdenum, and tungsten, is recommended. In particular, the base body of a stent may contain a biocorrodable magnesium alloy or be composed entirely of the same.

“Alloy” is understood herein to mean a metallic structure having magnesium, iron, zinc, or tungsten as its main component. The main component is the alloy component having the highest proportion by weight in the alloy. A proportion of the main component is preferably greater than 50% by weight, and in some embodiments greater than 70% by weight. Other weight compositions will also be suitable.

The composition of alloys of the elements magnesium, iron, zinc, or tungsten should be selected so as to be biocorrodable. Within the meaning of the invention, “biocorrodable” refers to alloys for which, in a physiological environment, degradation occurs which ultimately results in the entire implant or the part of the implant formed from the material losing its mechanical integrity. Synthetic plasma as specified according to EN ISO 10993-15:2000 for biocorrosion testing (composition: NaCl 6.8 g/l, CaCl₂ 0.2 g/l, KCl 0.4 g/l, MgSO₄ 0.1 g/l, NaHCO₃ 2.2 g/l, Na₂HPO₄ 0.126 g/l, NaH₂PO₄ 0.026 g/l) is a suitable test medium for testing the corrosion behavior of a given alloy. A sample of the alloy to be tested is accordingly stored together with a defined quantity of the test medium in a sealed sample container at 37° C. At time intervals of a few hours to several months, depending on the expected corrosion behavior, the samples are removed and investigated in a known manner for signs of corrosion. The synthetic plasma according to EN ISO 10993-15:2000 corresponds to a medium similar to blood, and therefore provides the possibility for reproducibly representing a physiological environment within the meaning of the invention.

The term “corrosion” herein refers to the reaction of a metallic material with its environment whereby, when the material is used in a component, a measurable change in the material causes impairment of the function of the component. In the present context a corrosion system is composed of the corroding metallic material and a liquid corrosion medium whose composition reproduces the conditions in the physiological environment, or which is a physiological medium, in particular blood. Material factors which influence the corrosion include the composition and pretreatment of the alloy, microscopic and submicroscopic inhomogeneities, boundary zone characteristics, temperature and stress state, and in particular the composition of a layer covering the surface. With regard to the medium, the corrosion process is influenced by conductivity, temperature, temperature gradients, acidity, volume-surface ratio, concentration difference, and flow velocity.

Suitable biocorrodable metallic implant materials include (but are not limited to) those having an element from the group of alkali metals, alkaline earth metals, iron, zinc, and aluminum as their main component. Alloys based on magnesium, iron, and zinc are described as being particularly suitable. Secondary components of the alloys may include manganese, cobalt, nickel, chromium, copper, cadmium, lead, tin, thorium, zirconium, silver, gold, palladium, platinum, silicon, calcium, lithium, aluminum, zinc, and iron. Furthermore, embodiments of the invention may also use a biocorrodable magnesium alloy, with one example having a proportion of magnesium >90%, yttrium 3.7-5.5%, rare earth metals 1.5-4.4%, and the remainder <1%. It has been discovered that this example composition is particularly suited for manufacturing an endoprosthesis, for example in the form of a self-expanding or balloon-expandable stent.

Various aspects of invention embodiments are explained in greater detail below with reference to one example embodiment and with reference to the Figures.

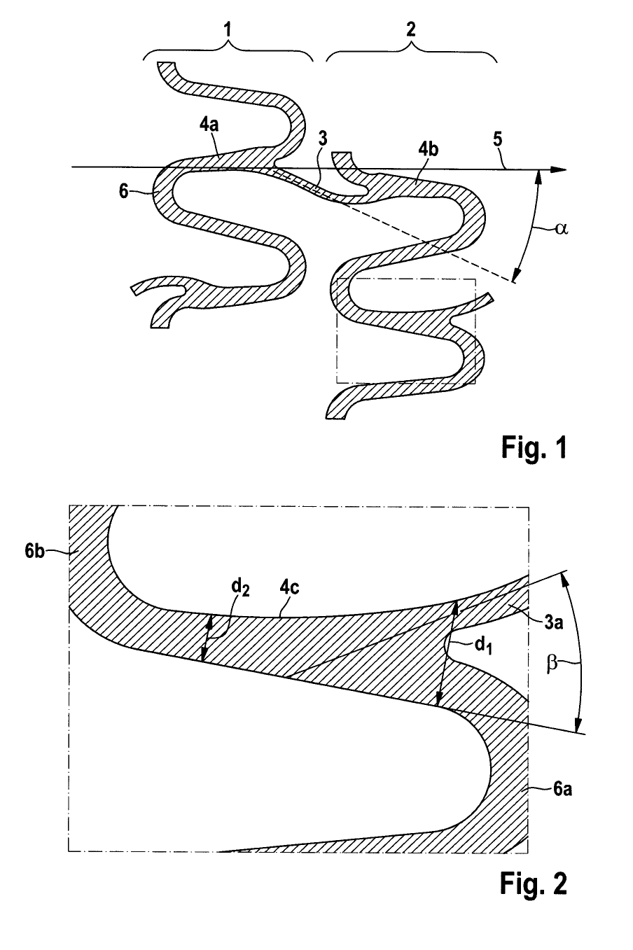

FIG. 1 shows a section of a base body of a stent according to the one example invention embodiment. Illustrated are sections of two successive support structures 1 and 2 which are joined together by a connector 3. The support structures 1 and 2 are composed of a sequence of diagonal elements 4 and arched elements 6. The connector 3 connects the two support structures 1 and 2 via the diagonal elements 4a and 4b. For this purpose the connector 3 is oriented in the longitudinal axis 5 and has an angle α of ≧0° and <70° with respect to the longitudinal axis. The connector 3 has a curved shape, and branches off from each of the diagonal elements 1 and 2 at an acute angle. Other angles α and elements 4 and 6 may be utilized in other invention embodiments, eith examples including (but not limited to) angles α >70°, elements 6 that are not arched.

FIG. 2 shows a section from FIG. 1, indicating the detailed shape of a diagonal element 4c to which the connector 3a is attached. The diagonal element 4c has an elongated shape with two opposite ends. At its first end the diagonal element 4c has a branching point at which the diagonal element directly branches into the connector 3a and the arched element 6a. At this branching point the diagonal element 4c has a diameter d₁. At its second end the diagonal element 4c has a connecting point at which the diagonal element 4c directly merges into the arched element 6b. At this connecting point the diagonal element 4c has a diameter d₂. In this embodiment, the diagonal element 4c has been designed in such a way that the ratio of d₁ to d₂>1, and in particular is between 1.25 and 2.5. It has been discovered that this ratio provides unexpected benefits and advantages in at least some applications. The diagonal element tapers continuously from its first end toward its second end. This tapering is not uniform, and is more pronounced in a region facing the first end of the diagonal element 4c than in a region facing the second end of the diagonal element. The connection of the connector 3a is thus supported over the entire length of the diagonal region 4c. Again, it has been discovered that this configuration provides unexpected benefits and advantages in at least some applications, with an example being favorable combination of mechanical strength to element mass and/or size. Other invention embodiments may utlize other tapering configurations, with one example being a generally uniform tapering from first to second ends. The connector 3a branches off from the diagonal element 4c at an angle β which is acute (β>0 and <90°). Other invention embodiments may utilize other tapering configurations, with one example being a generally uniform tapering from first to second ends, as well as other angles β.

As a result of the organic attachment of the connectors to the diagonal elements, the distribution of expansion and stress in the severely strained arched elements of the stent according to these invention embodiments is not influenced. This represents an important and beneficial improvement over the prior art, and ensures optimal utilization of material. In addition, the large-surface connector attachment assists in optimal transmission of force from one support structure to another. The axial orientation of the connectors and the acute-angle connection further result in an optimal use of space in the crimped state. Again, important benefits are achieved over the prior art.

It will be apparent to those skilled in the art that numerous modifications and variations of the described examples and embodiments are possible in light of the above teaching. The disclosed examples and embodiments are presented for purposes of illustration only. Therefore, it is the intent to cover all such modifications and alternate embodiments as may come within the true scope of this invention.

### LIST OF REFERENCE CHARACTERS

1 First support structure
2 Second support structure
3, 3a Connector
4a, 4b, 4c Diagonal element to which a connector is attached
5 Longitudinal axis
6, 6a, 6b Arched element
α Angle between the longitudinal axis and the connector
β Acute angle at which the connector branches off from the diagonal element
d₁ Diameter at the first end of the diagonal element
d₂ Diameter at the second end of the diagonal element

What is claimed is:

1. An expandable stent comprising a tubular base body with a lumen along a longitudinal axis, wherein the base body comprises:
a plurality of circumferential support structures which are successively positioned along the longitudinal axis and are each composed of a sequence of diagonal elements and arched elements; and
one or more connectors, wherein two successive circumferential support structures are joined together by at least one connector, and each connector is attached to one diagonal element of the two circumferential support structures to be connected;
the stent further characterized in that:
at least one diagonal element to which a connector is attached has an elongated shape with first and second opposite ends,
i) wherein the diagonal element at its first end has a branching point with a diameter d₁ at which the diagonal element directly branches into an arched element and a connector;
ii) wherein the diagonal element at its second end has a connecting point with a diameter d₂ at which the diagonal element directly merges into a further arched element; and
iii) wherein the ratio of d₁ to d₂>1, and the diagonal element is tapered from its first end to its second end.

2. The stent according to claim 1, characterized in that the connector branches off from the diagonal element at an acute angle.

3. The stent according to claim 1, characterized in that the connector has a curved shape.

4. The stent according to claim 1, characterized in that the connector is oriented in the longitudinal direction between the two circumferential support structures to be connected, and in the dilated state of the stent the connector forms an angle α of ≧0° and <70° with respect to the longitudinal axis.

5. The stent according to claim 1, characterized in that the ratio of d₁ to d₂ is between 1.1 and 5.

6. The stent according to claim 1, characterized in that the diagonal element to which a connector is attached is uniformly tapered from its first end toward its second end.

7. The stent according to claim 1, characterized in that the diagonal element to which a connector is attached is nonuniformly tapered from its first end toward its second end, the tapering being more pronounced in a region proximate the first end of the diagonal element than in a region proximate the second end.

8. The stent according to claim 1, characterized in that the base body comprises a metallic implant material.

9. The stent according to claim 1, characterized in that the base body contains a biocorrodable implant material.

10. The stent according to claim 1, characterized in that the base body contains a biocorrodable magnesium alloy.

11. The stent according to claim 1 wherein the connector forms an angle α ≧0° and <45° with respect to the longitudinal axis.

12. The stent according to claim 1 wherein the connector forms an angle α ≧0° and <25° with respect to the longitudinal axis.

13. The stent according to claim 1, wherein the ratio of d₁ to d₂ is between 1.2 and 3.

14. The stent according to claim 1, wherein the ratio of d₁ to d₂ is between 1.25 and 2.5.

15. The stent according to claim 1, wherein the base body is composed entirely of a biocorrodible metallic implant material.

16. The stent according to claim 1, wherein the base body is composed entirely of a biocorrodible magnesium alloy.

17. An expandable stent having a tubular base body made at least partially from a metallic implant material and having a lumen along a longitudinal axis, the base body comprising:
a plurality of circumferential support structures successively positioned along the longitudinal axis and each having a plurality of diagonal elements and a plurality of arched elements;
at least one connector joining two successive circumferential support structures together through attachment to a diagonal element of each of the two circumferential support structures, the connector oriented in the longitudinal direction between the two connected circumferential support structures wherein in a dilated state of the stent the connector defines an angle α of between about 0° and about 70° with respect to the longitudinal axis; and
wherein each of the diagonal elements to which a connector is attached has an elongated and tapered shape between first and second opposing ends, the first end having a branching point with a diameter d₁ at which a diagonal element directly branches into an arched element and the connector, and the second end having a connecting point with a diameter d₂ at which a diagonal element directly merges into one of the arched elements, the ratio of d₁ to d₂ between about 1.1 and about 5.

18. An expandable stent as defined by claim 17 and wherein:
the connector has a curved shape and branches off from the diagonal element at an acute angle;
α is between about 0° and about 45°; and,
the ratio of d₁ to d₂ is between about 1.25 and about 2.5.

19. An expandable stent as defined by claim 17 and wherein:
the base body is comprised of a biocorrodable material;
α is between about 0° and about 25°; and,
the ratio of d₁ to d₂ is between about 1.1 and about 3.

20. An expandable stent as defined by claim 17 and wherein:
the base body is made entirely of a biocorrodible magnesium alloy;
the connector branches off from the diagonal element at an acute angle, and,
the diagonal element to which a connector is attached is non-uniformly tapered from its first end toward its second end, the tapering being more pronounced in a region proximate the first end of the diagonal element than in a region proximate the second end.
